(19) United States
(12) Patent Application Publication
(10) Pub. No.: US 2010/0189727 A1
(43) Pub. Date: Jul. 29, 2010

(54) Masking Ligands For Reversible Inhibition Of Multivalent Compounds

(75) Inventors: Ulrich Rodeck, Philadelphia, PA (US); John Williams, Monrovia, CA (US)

Correspondence Address: RATNERPRESTIA, P.O. BOX 980, VALLEY FORGE, PA, 19482, US

(73) Assignee: Tegopharm Corporation, Washington Crossing, PA (US)

(21) Appl. No.: 12/633,102

(22) Filed: Dec. 8, 2009

Related U.S. Application Data
(60) Provisional application No. 61/120,657, filed on Dec. 8, 2008.
(60) Provisional application No. 61/147,611, filed on Jan. 27, 2009.

(51) Int. Cl. A61K 39/395; C07K 2/00; C07K 7/08; C07K 14/00; C07K 16/00; A61P 35/00; A61P 17/02

(52) U.S. Cl. 424/178.1; 530/300; 530/326; 530/324; 530/350; 530/391.1; 530/391.3

(57) ABSTRACT

Masking ligands for reversibly concealing the antigen-binding site of an antibody comprise epitopes of the antibody and a cleavable linker. Methods for making masking ligands comprise joining at least two copies of the epitope of an antibody to a cleavable polypeptide linker.

### CROSS REFERENCE TO RELATED APPLICATIONS

This application claims priority to U.S. Provisional Patent Application No. 61/120,657, filed Dec. 8, 2008, and U.S. Provisional Patent Application No. 61/147,611, filed Jan. 27, 2009. The contents of each of which are incorporated herein by reference, in their entirety and for all purposes.

### FIELD OF THE INVENTION

This application relates generally to the field of protein engineering and therapy. More particularly, the application relates to masking ligands that comprise antibody epitopes connected to a cleavable polypeptide linker, as well as complexes and compositions comprising the same.

### BACKGROUND OF THE INVENTION

Various publications, including patents, published applications, technical articles and scholarly articles are cited throughout the specification. Each of these cited publications is incorporated by reference herein, in its entirety.

Adverse events caused by biologically active compounds administered with either therapeutic or diagnostic intent remain a significant problem in the pharmacological management of many disease states including infections, inflammation, autoimmune syndromes and neoplasia. In many cases such adverse events occur due to interaction of the biologically active compound with normal cells and tissues, thus thwarting the intent of “targeted” therapy. In the case of protein reagents adverse events can result from the interactions of cell-associated receptors with pharmacologically active ligands. Monoclonal antibodies (mAbs) are an important class of such ligand/receptor systems. mAbs typically bind to antigens implicated in the pathogenesis of specific disease states and can affect cell survival through both immunological and non-immunological mechanisms.

Therefore, it is highly desirable to direct pharmacologically active compounds specifically to disease sites and to reduce reactivity of these compounds with normal tissue. Heretofore, this problem has been approached by targeting molecular entities, e.g., antigens, present at high levels in disease tissues compared with normal tissues. This approach is limited, because relevant target molecules may not be overexpressed at disease sites or may have a critical function in both normal and diseased tissues regardless of expression levels. For example, mAbs which react with cell surface receptors of the ErbB family are frequently used to treat tumors, but these mAbs also cause serious adverse effects affecting normal tissues that express the cognate antigens, such as the heart, gastrointestinal system and the skin (Chien, New England J. Med. 354: 789-790, 2006; Lemmens, et al., Circulation 116: 954-960, 2007; Pastore, et al., J. Investigative Dermatology 128: 1365-1374, 2008). In addition, adverse side effects frequently lead to non-compliance with mAb treatment (Boone, et al., Oncology 72: 152-159, 2007). Furthermore, systemic administration of mAbs for prolonged periods of time may be associated with increased risk of developing infections and/or neoplasia in treated patients (Jones and Loftus, Inflamm. Bowel Dis. 13: 1299-1307, 2007; Williams, Eur. J. Cancer Prevention 17: 169-177, 2008).

### SUMMARY OF THE INVENTION

The invention features masking ligands for reversibly concealing the antigen-binding site of an antibody. In general, the masking ligands comprise two copies of the epitope of the antigen to which the antibody specifically binds and a cleavable polypeptide linker joined to each copy of the epitope.

In some aspects, the masking ligand reversibly conceals each antigen-binding site of an antibody having more than one antigen binding site. At least one of the antigen binding sites has a different antigen specificity relative to the other antigen binding sites. In general, such masking ligands comprise a copy of the respective epitope for each antigen binding site of the antibody and a cleavable polypeptide linker joined to each copy of an epitope.

For masking ligands, the polypeptide linker preferably comprises at least one proteolytic enzyme recognition site, and the proteolytic enzyme is preferably a matrix metalloprotease, with matrix metalloprotease 2 and matrix metalloprotease 9 being highly preferred. The polypeptide linker preferably comprises SEQ ID NO:5.

The epitope of the masking ligand can comprise at least one amino acid mutation that decreases the affinity of the antibody for the epitope. The epitope can comprise the amino acid sequence of SEQ ID NO:7, SEQ ID NO:8, SEQ ID NO:9, or SEQ ID NO:10.

The antibody can specifically bind to epidermal growth factor.

The invention also features complexes comprising a masking ligand noncovalently bound to the antigen binding site of an antibody. The masking ligand preferably comprises a cleavable polypeptide linker. The antibody can comprise a detectable label, and/or at least one post-translational modification. The complex can be provided in a composition with a pharmaceutically acceptable carrier.

The invention also features methods for preparing a multivalent masking ligand. In general, the methods comprise joining at least two copies of the epitope of an antibody to a cleavable polypeptide linker comprising at least one proteolytic enzyme recognition site. Each copy of the epitope can be chemically modified to include a moiety that can bind to the polypeptide cleavable linker. Preferably, the cleavable polypeptide linker has the amino acid sequence of SEQ ID NO:5.

The invention also features methods for treating a subject in need thereof. In general, the methods comprise administering to a target tissue in the subject an effective amount of a complex comprising a masking ligand noncovalently bound to an antibody. The complex can be provided in a composition with a pharmaceutically acceptable carrier. The target tissue is preferably a tissue that expresses a proteolytic enzyme capable of cleaving a cleavable polypeptide linker of the masking ligand. The antigen binding site becomes available when the masking ligand dissociates from the antigen binding site of the antibody at the target tissue after the proteolytic enzyme cleaves the cleavable polypeptide linker. The target tissue can be a tumor, ulcer, wound, or a tissue that is inflamed.

### BRIEF DESCRIPTION OF THE DRAWINGS

FIG. 1 shows a schematic diagram of a bivalent masking ligand. FIG. 1A shows identical linked masks. FIG. 1B shows nonidentical linked masks.

FIG. 2 shows a schematic of an antibody whose antigen-binding site is concealed by a multivalent masking ligand with two identical masks.

FIG. 3 shows a model of in vivo activation of masked mAbs to EGFR in target tissue.

FIG. 4 shows sequences of multivalent masking ligands reactive with Cetuximab and/or Matuzumab/425. FIG. 4A shows two EGFR masks based on the epitope in EGFR domain III connected by a linker (underlined) having a MMP-9 proteolytic site (bold); (SEQ ID NO. 7). This mask will bind with high affinity to Cetuximab (C225) or Matuzumab (derived from murine mAb425) (see Example 1). FIG. 4B shows two EGFR masks based on the epitope in EGFR domain III connected by a linker (underlined) having a MMP-9 proteolytic site (bold) and having two mutations —P for S (bold, larger font). This mask will bind to Cetuximab more weakly than the mask in A; (SEQ ID NO. 8). FIG. 4C shows two EGFR masks based on the epitope in EGFR domain III connected by a linker (underlined) having a MMP-9 proteolytic site (bold) and having two mutations -E for H (bold, larger font). This mask will bind to Matuzumab/425 more weakly than the mask in A; (SEQ ID NO. 9). FIG. 4D shows two EGFR masks based on the epitope in EGFR domain III connected by a linker (underlined) having a MMP-9 proteolytic site (bold) and having mutation -E for H in to one mask and mutation P for S in the other (bold, larger font). This mask can be used to connect Cetuximab and Matuzumab/425; (SEQ ID NO. 10).

FIG. 5 shows schematic reaction sequences illustrating chemical construction of a linker containing a protease recognition sequence.

FIG. 6 shows a schematic of tetrameric complex of two different antibodies is linked by a multivalent masking ligand with distinct masks.

FIG. 7 shows a schematic of tetrameric complex of two antibodies created by cross-linking the masking ligands for each antibody.

FIG. 8A shows a schematic of generic masking ligand and diabody blocking antigen binding sites of an antibody, and FIG. 8B shows a schematic of tetrameric complex of two different antibodies linked by a generic multivalent masking ligand.

FIG. 9 shows the production in insect cells and detection of the TGP1 masking agent. SDS gel electrophoresis of TGP1 as eluted from the Ni-His column and stained with coomassie blue are shown. A single band of the predicted molecular mass was evident in both, media as well as fractions denoted 5 and 6 eluted from the column. Lane (1)-medium; lane (2) column flowthrough; lane (3)-wash; lanes (4-7)-eluted fractions; lane (8)-denoted M for size marker.

FIG. 10 shows the detection of the masking agent (TGP1) by immunoblot analysis using mouse anti-His6 mAb. TGP1 consists of tandem EGFR domainIII fragments linked by an MMP9 sensitive cleavage site as shown in FIG. 4A.

FIG. 11 shows that Cetuximab (C225) binds to TGP1 immobilized on Ni-His beads. Ni-His columns preloaded with TGP1 were used to capture Cetuximab which was then eluted and detected by coomassie blue staining after gel electrophoresis under non-denaturing conditions. Control antibody recognizing human LewisY (15-6A) was not captured.

FIG. 12 shows that Cetuximab (C225) binds to TGP1 immobilized on Ni-His beads as determined by immunoblot analysis. Protein species consistent in molecular mass with heavy and light Ig chains are recovered in the C225 eluate whereas in the case of control 15-6A no antibody-derived protein species are evident.

FIG. 13 shows the cleavage of the TGP1 mask by MMP9. Coomassie blue staining of protein species resolved by SDS-PAGE is shown. Lane (1)-Control TGP1 in the absence of MMP9; lanes (2-4)-TGP1 treated with increasing concentration of MMP9 (25, 50, 150 ng) for 4 h at room temperature; lanes (5-7)-TGP1 treated with increasing concentration of MMP9 (25, 50, 150 ng) for 30 min at 37° C. TGP1 cleavage is demonstrated indicated by reduced abundance of the high molecular weight protein band and appearance of lower molecular weight species.

FIG. 14 shows digestion of TGP1/C225 complexes with MMP9. Approx 5 μg TGP was incubated with 20 μg C225 at 4° C. for 20 minutes prior to adding MMP9. Complete cleavage of TGP1 (comigrating with TGP1 in lane 1) is evident.

FIG. 15 shows decreased binding of Cetuximab (C225) to MDA-MB-468 target cells as determined by FACS analysis and following preincubation with TGP1. Almost complete binding inhibition was achieved relative to unmasked antibody. Upper panel represent FACS histograms, lower panel shows mean fluorescence intensity (MFI) of peaks on histograms.

FIG. 16 shows decreased binding of 425 to MDA-MB-468 target cells as determined by FACS analysis and following preincubation with TGP1. Almost complete binding inhibition was achieved relative to unmasked antibody. Legend as in FIG. 15.

FIG. 17 shows MMP9 cleavage of the Cetuximab/TGP1 complex restores binding of TGP-1 masked Cetuximab to MDA-MB-468 cells.

FIG. 18 shows MMP9 cleavage of the Cetuximab/TGP1 complex restores binding of 425 to MDA-MB-468 cells.

### DETAILED DESCRIPTION OF THE INVENTION

The methods and compositions described herein facilitate molecular interactions between therapeutically active antibodies or other therapeutic protein molecules and targeted cells at disease sites, while avoiding activity in normal tissues. To accomplish this, the therapeutic molecules are “inactivated” by reversibly concealing their epitope/ligand binding sites and, thus, blocking antigen recognition and engagement in normal tissues. However, molecular events that occur predominantly at disease or target sites are exploited to restore pharmacological activity to the therapeutic antibody or protein, which then become free to bind their cognate epitope or ligand.

This approach is referred to herein as masking or concealing the antigen-binding site of an antibody, and the recombinant protein is referred to as a “mask.” Because masking the antigen-binding site of an antibody can potentially interfere with the antibody's capacity to bind to its antigen in disease tissue, a mechanism is needed to activate the antibody, and this preferably will occur at or proximal to the disease site.

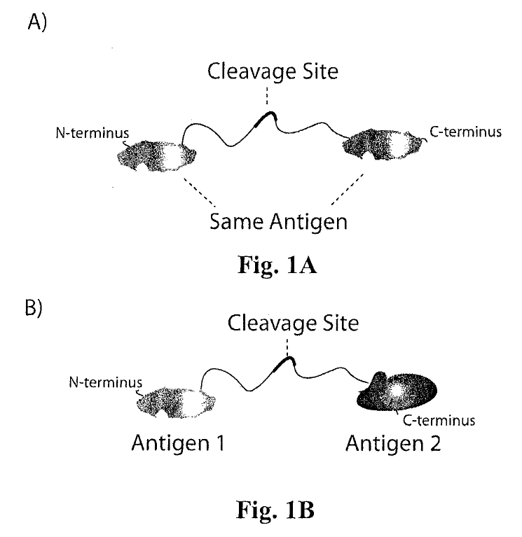

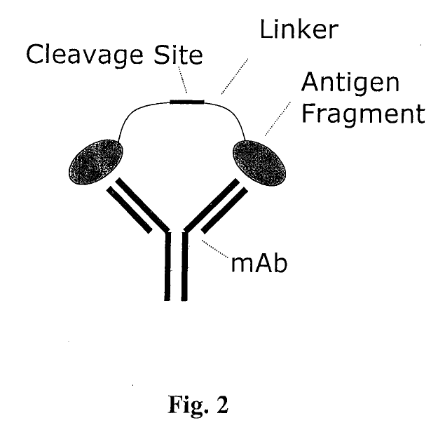

Accordingly, the invention features masking ligands for reversibly concealing the antigen-binding site of an antibody. Generally, the masking ligands comprise the epitope of the antigen to which the antibody specifically binds, and preferably comprise at least two copies of the epitope, with each copy connected to the other by way of a cleavable polypeptide linker. Each copy of the epitope interacts with one of the antigen binding sites on the antibody. Such antibodies are preferably bivalent or otherwise comprise at least two antigen binding sites. Examples of bivalent masks are illustrated in FIGS. 1 and 2.

The linker can comprise a polypeptide sequence that is capable of being cleaved by a specific proteolytic enzyme, preferably such an enzyme that is highly expressed in the disease tissue. Some preferred examples of such enzymes include matrix metalloproteinases (MMP), including any of MMP 1-28. Other suitable enzymes include prostate-specific antigen (PSA, a serine protease), ADAM proteases (disintegrin and metalloprotease), and plasminogen activators. Generally, it is preferred that the enzyme be a disease-associated protease, for example, a protease expressed at elevated levels at disease sites. In some preferred aspects, the protease is expressed at elevated levels by a cancer, including prostate cancer. Once the linker is cleaved, the affinity of the antibody for the separated masks decreases, and the masks dissociate from the antibody at the site of the disease tissue. The unmasked antibodies become “activated,” and can preferably preferentially bind their cognate antigens on the target cells in the disease tissue aided by diffusion of the dissociated masks, which can then pass into the bloodstream, and be metabolized and/or excreted.

The linker can comprise a proteolytic cleavage site that will be digested by one or more enzymes present in the target tissue. In some preferred aspects, the proteolytic enzyme will be specifically expressed only in the target tissue affected by the disease. For example, prostate-specific antigen is a proteolytic enzyme found specifically in the prostate, which recognizes and cleaves a specific amino acid sequence (Khan and Denmeade, The Prostate 45: 80-83, 2000; DeFeo-Jones, et al., Mol. Cancer. Therap. 1: 451-459, 2002). In some aspects, the cleavage site will be recognized by proteolytic enzymes that are more highly expressed in the disease tissue than in normal tissue, such as legumain and matrix metalloproteinases, which perform essential functions in tumor formation, invasion, and metastasis (Liu, et al., Cancer Research 63: 2957-2964, 2003; Egeblad and Werb, Nat. Rev. Cancer 2: 161-174, 2002; Overall and López-Otin, Nat. Rev. Cancer 2: 657-672, 2002). In particular, MMP-9 and MMP-2 are associated with all stages of tumor progression and play a role in invasiveness in a wide variety of solid tumors (Kline, et al., Mol. Pharmaceutics. 1: 9-22, 2004). In some preferred aspects, the polypeptide linker comprises SEQ ID NO:5.

The selection and design of appropriate cleavage sites can be guided by the prevalence of the corresponding proteolytic enzyme(s) at the disease target site. Databases, e.g., MEROPS, and publications are available that list the cleavage recognition sequences for known proteolytic enzymes and describe the use of degradomics for identifying proteases and their specific substrates (López-Otin and Overall, Nature Reviews Molecular Cell Biology 3: 509-519, 2002; Doucet, et al, Mol. Cell. Proteomics 7: 1925-1951, 2008). Methods have also been disclosed for optimizing the substrates for MMP-1 and MMP-9 (McGeehan, et al., J. Biol. Chem. 269: 32814-32820, 1994).

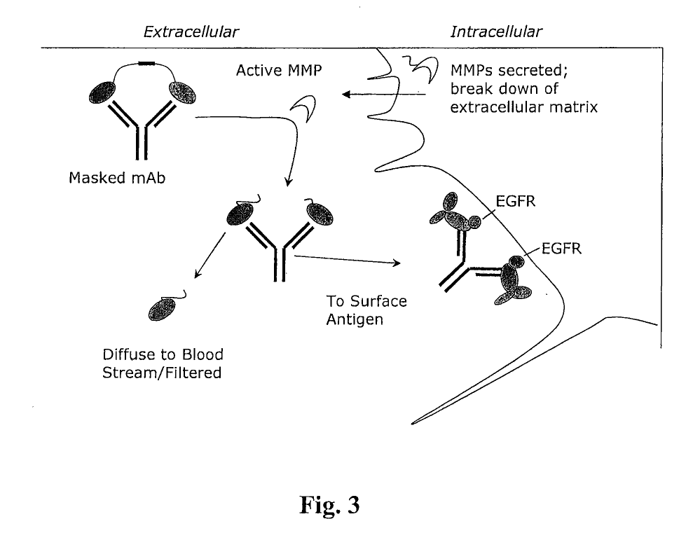

An exemplary model of a bivalent mask having a MMP cleavage site and capable of concealing the binding sites of a monoclonal antibody (mAb) specific for EGFR is shown in FIG. 3. In this model, MMPs which are highly expressed in tumor tissue are secreted by the tumor cells and released into the tumor microenvironment. The MMPs recognize and cleave the proteolytic site in the linker connecting the masks bound to the therapeutic mAb. As a result, the affinity of the cleaved, now monovalent, masks for the mAb is reduced and the masks dissociate from the antibody. The epitope binding sites of the antibody are now “unmasked” and capable of binding to the antigen, EGFR, on the tumor cells.

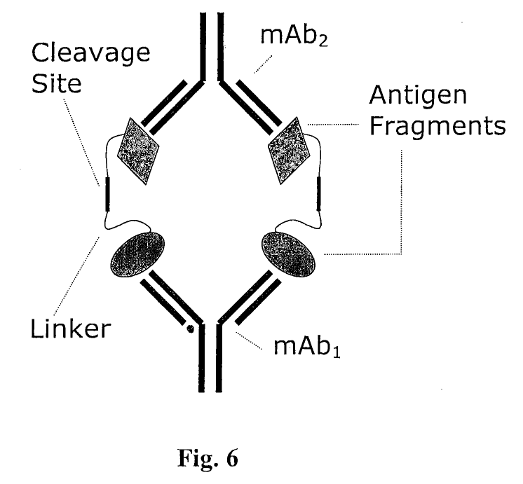

The masking ligands can be used on any type of antibody, and any isotype and idiotype of antibody. The ligands can, for example, be used on polyclonal antibodies, monoclonal antibodies, single chain antibodies, antibody fragments such as Fab's and Fv's, phage-displayed antibodies, and the like. Additionally, the antibodies can be engineered to have multiple specificities. For example, the antibody can comprise different heavy and light chain pairs which each comprising an antigen binding site that specifically binds to a different antigen than the other antigen binding site(s). The antibodies thus are specific for more than one antigen (as distinct from reactive antigen binding sites that are cross-reactive with more than one antigen). Examples of such antibodies include diabodies. Similarly, mAbs that bind to identical or different epitopes of the same antigen can be linked by multivalent masks to enhance the therapeutic effect at the target tissue (FIGS. 1, 6). Because mAbs that recognize antigens in both affected and normal tissues are increasingly being used to treat malignant and inflammatory conditions, there is a risk of adverse side effects to is normal tissue.

Examples of suitable antibodies that can be used in the invention include, but are not limited to, antibodies that specifically bind to epidermal growth factor (EGFR), erbB2, prostate-specific membrane antigen (PSMA), insulin-like growth factor (IGFR), vascular endothelia growth factor (VEGF), VEGF receptor (VEGFR), CD₂₀, CD11A, CD25, tumor necrosis factor (TNF), TNF-α, TNF-α receptor and carcinoembryonic antigen (CEA). Thus, in some aspects of the invention, antibody binding to antigens in normal tissue is reduced by non-covalently binding to the antibody a separate, recombinant protein containing the epitope specifically recognized by the antibody. The term epitope herein means the site on an antigen to which an antibody binds; the epitope can be in its native conformation, or can be in an intermediate configuration, or in a linear configuration such as a peptide mimic or mimotope.

The epitope can comprise entire (whole) antigen to which the antibody's antigen binding site specifically binds. The epitope can comprise only the section(s) of the antigen to which the antibody's antigen binding site specifically binds, for example, the epitope can be a fragment of the antigen. The epitope can be fused with other amino acids, polypeptides, or proteins. The epitope can be chemically modified, and the chemical modifications can decrease the affinity of the antibody for the epitope in the mask. For polypeptide epitopes, the epitope can comprise one or more mutations, and the mutations can decrease or otherwise weaken the affinity of the antibody for the epitope in the mask, and in turn facilitate dissociation. The mutations can, but need not, be to conservative amino acids. The mutations can be additions or deletions. The mutations need not be to those amino acids to which the antigen binding site specifically interacts, although the mutations can affect the orientation of such amino acids by altering the structure of the epitope.

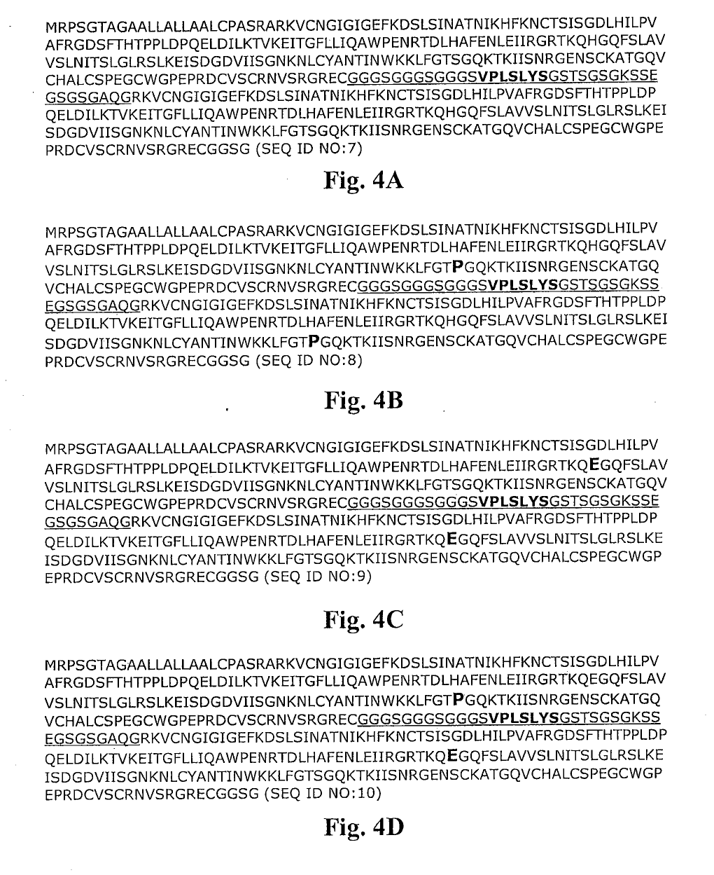

For example, for Cetuximab and Matuzumab/425, mutated mask sequences are shown in FIG. 4B-D, which have weaker affinities for these mAbs than the native EGFR domain III epitopes shown in FIG. 4A (Kamat, et al., Cancer Biol. And Ther. 7:726-33, 2008). Upon cleavage of the linker, the masks will dissociate from the antibodies and wild-type EGFR domain III antigen expressed on the target cells of tumor cells will be preferentially bound with comparatively higher affinity.

In some preferred aspects, the epitope comprises SEQ ID NO:7, SEQ ID NO:8, SEQ ID NO:9, or SEQ ID NO:10. The epitope can also consist essentially of, or consist of, these sequences.

These same concepts and techniques can also used to prepare masks for ligand is binding sites on other, non-antibody therapeutic proteins. Ligands are molecules or molecular groups that bind to another chemical entity to form a complex. In some aspects, two or more identical or distinct therapeutic proteins can be linked together with a multivalent masking ligand for enhanced delivery of the therapeutics to the target site.

The multivalent masking ligands non-covalently and reversibly conceal the antigen-binding site or other functional site of an antibody (i.e., complement binding site or Fc receptor). The masking ligand can be comprised of at least two masking moieties and a cleavable linker.

The invention includes masking complexes which are non-covalent in nature, i.e., do not affect the molecular composition of the therapeutic or diagnostic compounds, e.g., antibodies themselves. Thus, the masking complex described herein represent a targeted release principle rather than a permanent modification or molecular alteration of a preexisting drug. This characteristic sets this invention apart from similar approaches in the field focusing on proteolytic cleavage of single protein sequences containing the masking moiety and the target binding moiety within the same macromolecule. In the latter case, the molecular composition of the therapeutically active principle is permanently altered.

The epitope masking technology described herein may depend on detailed structural knowledge of the antigen/antibody interface via the antibody complementarity determining regions (CDR). This information is available for most therapeutic antibodies that are either approved for clinical use or are in clinical trials. These include, but are not limited to, Cetuximab, Matuzumab, Panitumumab, Trastuzumab, Pertuzumab, Rituximab, Bevacizumab, Gemtuzumab ozogamicin, Alemtuzumab, Ibritumomab tiuxetan, Tositumomab, Natalizumab, Certolizumab pegol, Etanercept, Adalimumab, Infliximab, Eculizumab, Efalizumab, Ranibizumab, Omalizumab, Arcitumomab, Imciromab pentetate, Capromab pendetide, Basiliximab, Palivizumab, Nofetumomab, Daclizumab, Fanolesomab, and Ustekinemab.

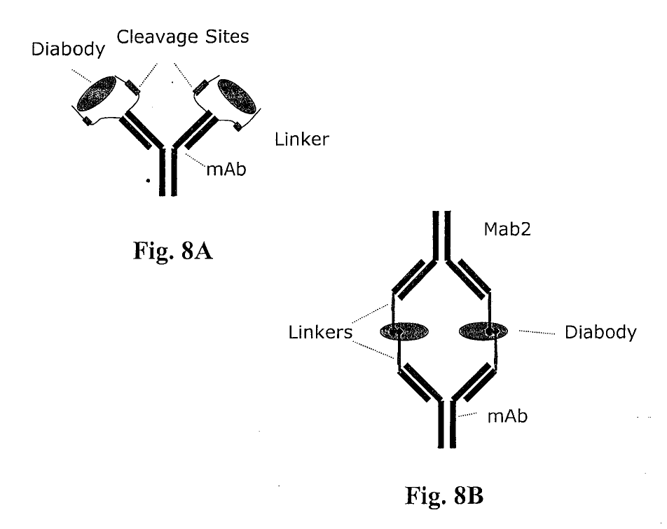

However, a generic method can also be applied to reversibly conceal antigen binding sites on any monoclonal antibody, even when the epitope structure is unknown. In some aspects of this method, ligands can be constructed which recognize the framework regions that are common to all antibodies of a particular isotype. For example, most monoclonal antibodies currently used in cancer therapy are of the human IgG1 isotype. Although these mAbs each have unique and distinct CDRs for antigen recognition, all human IgG1 molecules share a common framework in which the CDRs are embedded. A multivalent, cleavable masking ligand that binds to these shared framework regions and conceals the epitope binding site of the mAb is used to form a tetrameric (or larger) complex in which the CDRs of one mAb are juxtaposed to the CDRs of another mAb (FIG. 8). In this way, the CDRs of each mAb are concealed and cannot bind to native antigen on target cells.

For the generic ligand, the mask itself may comprise the CDR of a monoclonal antibody recognizing the common framework residues contained within the variable domain of a particular immunoglobulin isotype, for example, human IgG1. Proteolytic cleavage sites are selected and engineered into the linker as described above, and proteolytic cleavage in the target tissue dissociates the complex, thus releasing “active” therapeutic mAbs. An example of a generic masking ligand for IgG1 is presented in SEQ ID NO:6.

Alternatively, the N-termini of the CDR framework regions can be derivatized by adding tags, e.g., H is or FLAG® (Sigma-Aldrich, St. Louis, Mo.). The tags provide sites for reversibly attaching generic masking ligands with proteolytic sites. The attached ligands are designed to bind to a diabody, which results in masking of the antigen binding site of the mAb (FIG. 8A). Diabodies are a new class of small bivalent and multi-specific antibody fragments, which are described in detail in Kortt A A et al. (2001) Dimeric and trimeric antibodies: high avidity scFvs for cancer targeting Biomol. Eng. 18(3):95-108. Diabody binding to generic masking ligands can also be used to form complexes of masked mAbs, as shown in FIG. 8b. An example of this type of generic masking ligand is described in Example 8.

The masking ligands can be used with antibodies having C-terminal modifications such as those that modify antibody efficacy and effector functions, carry a drug or a nucleotide, or carry an enzyme to activate a prodrug. The masking principle is independent of and effective for all known Fc, i.e., C-terminal antibody, modifications.

In some aspects of the invention, recombinant mask proteins are produced that mimic the natural epitope(s) which binds to the particular mAb(s) selected for therapy. For example, a multivalent masking ligand comprising two masks having the sequence of the natural epitope for EGFR domain III, (SEQ ID NO: 7), connected by a linker containing a proteolytic site for MMP-9 is shown in FIG. 4A. These masks contain epitopes that bind both Cetuximab and Matuzumab/425, two different mAbs for ErbB1 that are used to treat various epithelial cancers (based on Kamat, et al., Cancer Biology and Therapy 7: 726-733, 2008, and Li, et al., Cancer Cell 7: 301-311, 2005). FIG. 4D shows a multivalent masking ligand (SEQ ID NO:10) with one modified mask for Cetuximab and one modified mask for Matuzumab/425. This hetero-mask links the two therapeutic antibodies together and allows for their simultaneous delivery to the target site. The use of two distinct mAbs for the same protein has been shown to have synergistic efficacy in inhibiting cell proliferation and signal transduction in tumor cells (Kamat, et al., 2008). The same strategy can be used for other antibodies with known epitopes. For example, two mAbs that can neutralize human IGF1R are described in U.S. Pat. No. 7,217,796. One mAb binds to a structural domain believed to encompass amino acid residues 309 to 591. The other antibody binds to a domain within amino acid residues 191 to 309. As described above for EGFR, masks can be made to conceal the binding site on each of these antibodies and to link the two distinct antibodies together for simultaneous delivery.

The invention also provides complexes comprising the masking ligands described herein which are non-covalently bound to the antigen binding site of their cognate antibody. Such complexes can be used for treating subjects, or can be used diagnostically to detect specific biomarkers in a target tissue. The antibody can be linked to a second therapeutic agent, or can be linked to a detectable marker, such as a fluorescent tag, a dye, a radioisotope, or an enzyme. The antibody can be post-translationally or otherwise chemically modified. Modifications include glycosylation, acylation, alkylation, biotinylation, amidation, phosphorylation, pegylation, prenylation, glycation, and the like as known or otherwise used in the art.

Compositions containing such complexes and a pharmaceutically acceptable carrier and/or excipients are also provided. Such compositions can be used to deliver therapeutic antibodies in an inactive form to a target tissue. The composition can be delivered to a mammalian subject in need by any suitable method, including oral, intravenous, topical, enteral, parenteral, and direct injection or direct application to a target site, and the like. Dosage can be optimized for the antibody or antibodies used and for each subject according to methods known in the art. Carriers include, without limitation, water, saline, buffered solutions and the like.

After the antibody-mask complex is administered to the subject, the antibodies will be unmasked through the appropriate molecular machinery in the subject's body, and are preferably retained in the target tissue where they engage their cognate antigen and can be imaged at that site based on the type of detectable marker employed.

The invention also features methods for making masking ligands. Generally, the methods comprise joining the epitopes with a cleavable polypeptide linker which comprises at least one proteolytic enzyme recognition site. Methods for making a masking ligand can also generally comprise the steps of providing at least two epitopes, providing a cleavable linker, and joining the epitopes through the cleavable linker. The epitopes can be joined to the linker according to the therapeutic application to which the mask will be used, or according to the chemical properties of the mask. For example, joining can be by adsorption, electrostatic interactions, charge complexation, ionic bonding, or covalent bonding, and can include the use of biomolecule tethers.

Masking proteins or peptides mimicking the target epitope with cleavable linkers can be produced through genetic engineering and production of recombinant protein, e.g., by recombinant cells using standard techniques and commercially available protein expression systems as described in Example 1. The proteins and peptides can also be chemically synthesized according to methods known in the art, such as t-Boc/Fmoc solid phase and solution phase technology.

A flexible linker containing a cleavage site specific for a particular enzyme is prepared by any appropriate method, e.g., as described in Example 2. A preferred linker has the amino acid sequence (SSGS)ₙ-VPLSLYS-(SSGS)ₘ (e.g., SEQ ID NO:5), wherein n=1-3 and m=1-3, and VPLSLYS is a proteolytic site for MMP-9. However, any linker sequence that allows for the correct conformation and function of the masks may be used. A number of appropriate linkers and methods for preparing them are described in U.S. Pat. No. 6,541,219.

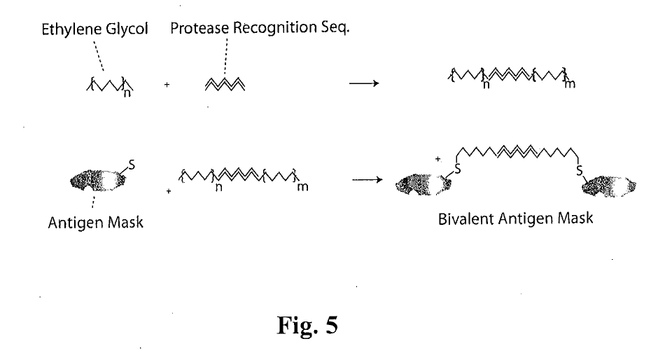

In some alternative aspects, polyethylene glycol can be the linker between the masks and the protease sites, as shown in FIG. 5 and described in detail in Example 2. Other methods may also be employed, e.g., as described in Krishnamurthy, et al., J. Am. Chem. Soc. 129:1312-1320, 2005.

Alternatively, a genetic construct can be produced that encodes a complete masking ligand, i.e., two or more mask peptides connected by one or more flexible linkers containing cleavage sites for a specific enzyme, e.g., SEQ ID NOs:1-4 or SEQ ID NOs:7-10. Recombinant cells/organisms comprising this construct are then used to produce the masking ligand. Standard methods of molecular biology are used to prepare the genetic construct and any appropriate expression system may be used to produce the masking ligands, for example, mammalian or insect cell systems or bacterial, yeast, or plant systems.

In some aspects, a bivalent masking construct is prepared that links two distinct (non-identical) masks (FIG. 1B). This construct can be used to mask and to link two different mAbs, forming a very stable tetravalent complex (FIG. 6). When the tetrameric complex is disrupted by proteolysis, two monovalent, weakly bound, mAb-epitope complexes are produced. The affinity between masks and antibody in the monovalent complex is greatly reduced and the masks dissociate readily, releasing “active” antibody. This phenomenon has been examined and confirmed with small molecules by Rao, et al., Science 280: 708-711, 1988.

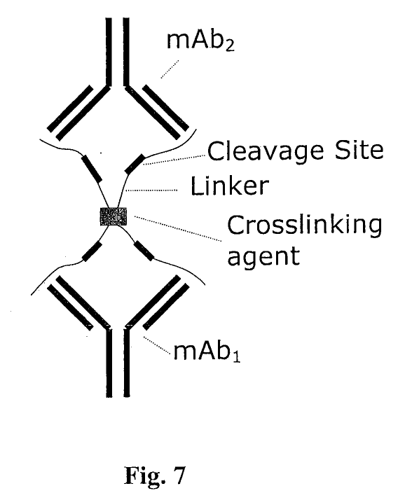

In some aspects, masked antibodies that are distinct are joined in a complex by cross-linking the masks, as shown in FIG. 7. In this way, two or more antibodies may be delivered simultaneously to a target site.

The invention also provides methods for reversibly occluding an antigen-binding site of an antibody comprising contacting a masking ligand such as those described herein with at least one antibody. The masking ligands will bind to their cognate antigen binding site, thereby forming masked antibody complexes. The masking ligands and antibodies can be combined in an appropriate stoichiometric ratio in an appropriate buffer, e,g., phosphate buffered saline (PBS). Depending on the linker length, a 1:1 or 2:2 antibody:antigen complexes can be formed.

The invention also provide methods for treating a subject in need thereof. Generally, the methods comprise administering to the subject an effective amount of a complex comprising a masking ligand and an antibody, such as the complexes described and/or exemplified herein. The complex can be administered in a pharmaceutically acceptable carrier. The methods are preferably adapted for treating a condition in the subject characterized by the expression of EGFR. Examples of such conditions include colorectal carcinoma (Frédéric Bibeau, F et al. (2006) Assessment of epidermal growth factor receptor (EGFR) expression in primary colorectal carcinomas and their related metastases on tissue sections and tissue microarray. Virchows Arch. 449(3): 281-287), non-small cell lung cancer (Dacic, S et al. (2006) Significance of EGFR Protein Expression and Gene Amplification in Non-Small Cell Lung Carcinoma Am. J. Clin, Pathol. 125(6):860-865), pancreatic carcinoma (Dancer, J et al. (2007) Coexpression of EGFR and HER-2 in pancreatic ductal adenocarcinoma: a comparative study using immunohistochemistry correlated with gene amplification by fluorescencent in situ hybridization. Oncology Reports 18:151-155; and, Chadha, K S et al. (2006) Activated Akt and Erk expression and survival after surgery in pancreatic carcinoma. Ann. Surg. Oncol. 13(7) 933-9), head and neck squamous cell carcinoma is (Ang, K K et al. (2002) Impact of epidermal growth factor receptor expression on survival and pattern of relapse in patients with advanced head and neck carcinoma. Cancer Research 62:7350-6; Sok, J C et al. (2006) Mutant epidermal growth factor receptor (EGFRvIII) contributes to head and neck cancer growth and resistance to EGFR targeting. Clin. Cancer Res. 12:5064-73; and, Dassonville, O et al. (1993) Expression of epidermal growth factor receptor and survival in upper aerodigestive tract cancer. J. Clin. Oncol. 11:1873-8), breast cancer (Milanezi, F et al. (2008) EGFR/HER2 in breast cancer: a biological approach for molecular diagnosis and therapy. Expert Rev. Mol. Diagn. 8(4):417-34), ovarian cancer (Maihle, N J et al. (2002) EGF/ErbB receptor family in ovarian cancer. Cancer Treat. Res. 107:247-58), bladder cancer, and glioma.

The effective amount of the complex may be dependent on any number of variables, including without limitation, the species, breed, size, height, weight, age, overall health of the subject, the type of formulation, the mode or manner or administration, the type and/or severity of the particular condition being treated, if the condition is cancer, it may depend on the stage of cancer, the extent of metastasis, and the like. The appropriate effective amount can be routinely determined by those of skill in the art using routine optimization techniques and the skilled and informed judgment of the practitioner and other factors evident to those skilled in the art. Preferably, a therapeutically effective dose of the complex will provide therapeutic benefit without causing substantial toxicity to the subject.

Toxicity and therapeutic efficacy of the complex can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents or compositions which exhibit large therapeutic indices are preferred. The data obtained from cell culture assays and animal studies can be used in formulating a range of dosage for use in the subject. The dosage of such agents or compositions lies preferably within a range of circulating concentrations that include the ED₅₀ with little or no toxicity. The dosage may vary within this range depending upon the dosage form employed and the route of administration utilized.

For any mask-antibody complex used in the methods of the invention, the therapeutically effective dose can be estimated initially from in vitro assays such as cell culture assays. For example, a dose can be formulated in animal models to achieve a circulating plasma concentration range that includes the IC₅₀ as determined in cell culture. Such information can be used to more accurately determine useful doses in a specified subject such as a human. The treating physician can terminate, interrupt, or adjust administration due to toxicity, or to organ dysfunctions, and can adjust treatment as necessary if the clinical response is not adequate in order to improve the response.

In some aspects, the dose of complex administered to the subject can also be measured in terms of total amount of drug administered per day. Treatment can be initiated with smaller dosages that are less than the optimum dose of angiocidin, followed by an increase in dosage over the course of the treatment until the optimum effect under the circumstances is reached. If needed, the total daily dosage may be divided and administered in portions throughout the day. The complex can may be administered alone or in combination with another active ingredient.

The following examples are provided to describe the invention in greater detail. The examples are intended to illustrate, not to limit, the invention.

### Example 1

### A Multivalent Cleavable Masking Ligand Genetically Engineered to Conceal the Antigen Binding Site of Cetuximab (C225)and Matuzumab/425

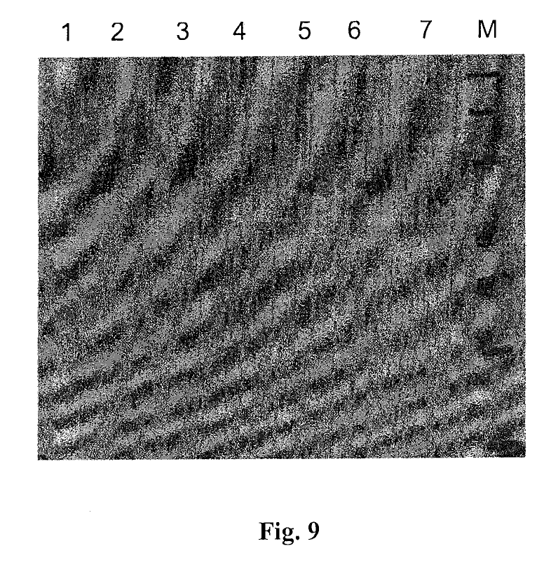

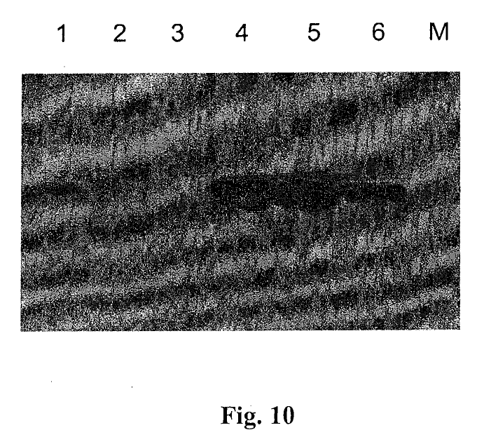

All constituent parts of the multivalent masking ligand hereafter referred to as TGP1 were expressed from one recombinant gene (FIG. 4A). A single protein comprising, in order, a Cetuximab/Matuzumab(425) binding mask (EGFR domain III epitope), a glycine-serine linker containing an MMP-9 cleavage site (GGGSGGGSGGGSVPLSLYSGSTSGSGKSSEGSGSGAQG) (SEQ ID NO:11) and a second Cetuximab/Matuzumab(425) binding mask were manufactured by automated chemical DNA synthesis. A polyhistidine purification tag (6-His) was included at the C-terminal end of the recombinant gene construct to aid in the purification of the recombinant protein. Although the two masks in this example are identical, they may also be distinct in order to link two distinct antibodies as described in the legend to FIG. 4. The recombinant gene was then ligated into the baculotransfer expression vector (pVL1392 (Invitrogen)) which was added with Baculogold™ linearized baculovirus DNA (Pharmingen) and Cellfectin® (Invitrogen) to the Spodoptera frugiperda cell line, sf9 (BD Biosciences). Supernatants containing recombinant baculovirus (recBV) were harvested and stored at 4° C. The viral stock was titered using High Five™ insect cells (Invitrogen). Specifically, 24 hours after infection with viral stock the cells were is examined by light microscopy for signs of infection such as cell rounding, detachment and failure to divide. Optimal titers were defined as 100% of the cells showing signs of infection. The recombinant multivalent masking ligand was produced in “High Five” cells infected with 5-10 infectious units per cell and it was secreted and accumulated in the supernatant, which was collected and centrifuged to remove cell debris. Clarified supernatant was equilibrated to a buffer containing 250 mM NaCl, 5 mM imidazole, a protease inhibitor cocktail (set III, CalBiochem) and recombinant protein purified by affinity chromatography specific for binding the polyhistidine tag (Ni-His column). Gel electrophoresis of the material eluted from the Ni-His column by a buffer containing high concentrations of imidazole (300 mM) revealed a single band of the expected molecular mass in lanes 4-7 (FIG. 9). As expected, proteins in this band contained the His-tag as evidenced by reactivity in immunoblot analysis with a mouse anti-His6 antibody (Genscript) detected by goat HRP conjugated anti-mouse IgG (FIG. 10). Fractions containing TGP1 were treated with 1 mM DTT, and dialysed overnight at 4° C. against 50 mM tris pH 7.5, 150 mM NaCl.

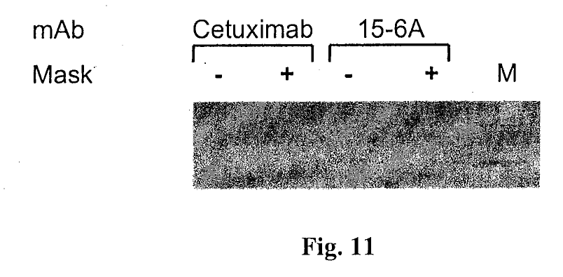

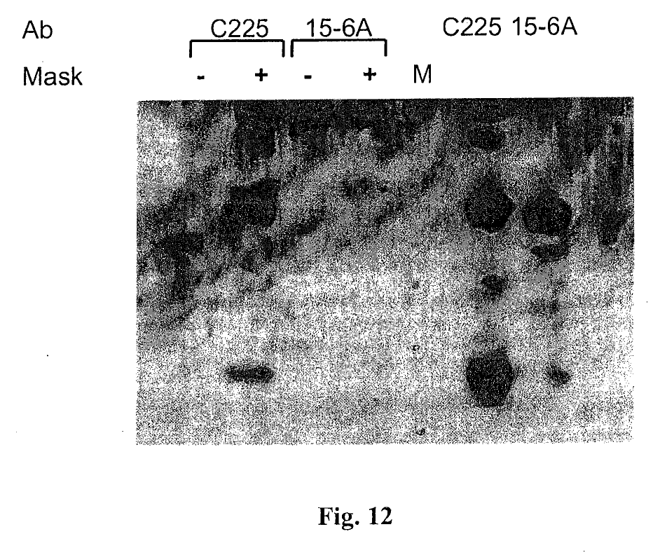

The purified TGP1 protein bound to a Ni-His resin was used to immobilize Cetuximab (FIGS. 11 and 12). Ten mL of harvested media, containing approx 4 μg TGP1 was loaded onto 150 mL Ni-His beads, which were divided into 2 columns. In addition and for control purposes two columns were packed with 50 L Ni-His beads without TGP1. Columns were washed with 50 mM tris pH 8.0, 250 mM NaCl, 40 mM imidazole, 20% glycerol. 20 μg mAb C225 or control antibody recognizing human LewisY (15-6A) in the above buffer were applied to each column at room temperature for 10 minutes, followed by washing with the same buffer. Bound proteins were eluted with 100 μL of buffer containing 300 mM imidazole. Results shown are of a Coomassie blue stained gel of eluents in SDS dye buffer without DTT to preserve native antibody configuration; M denotes molecular weight markers. An aliquot of 20 μL of each eluent was run on a SDS gel for staining and for transfer to nitrocellulose. Cetuximab bound to TGP1 whereas negative control antibody 15-6A did not. The 15-6A antibody recognizes the carbohydrate antigen human LewisY (15-6A; Rodeck et al., Hybridoma 6:389-401, 1987) which is not part of the recombinant TGP1 mask produced in insect cells.

Results shown are of eluents electrophoresed in SDS dye buffer without DTT to preserve native antibody configuration (FIG. 11). Binding of Cetuximab to TGP1 was specific, as the control antibody (15-6A) was not retained by TGP1. A single protein species consistent with Cetuximab is evident upon native gel electrophoresis whereas, under denaturing conditions, several protein species consistent with heavy and light chain were recovered in the Cetuximab eluate but not the control 15-6A eluate (FIG. 12). These protein species corresponding in size to heavy and light chains were recognized by rabbit anti human IgG followed by a mixture of peroxidase conjugated goat anti-rabbit and anti-mouse IgG (FIG. 12). As expected, no murine 1 g fragments were detected in the eluate from the TGP1 Ni-His column in case of negative control antibody 15-6A. Lanes denoted C225 and 15-6A to the right of Molecular weights markers (M) are of stock antibodies not passed over the Ni-His column.

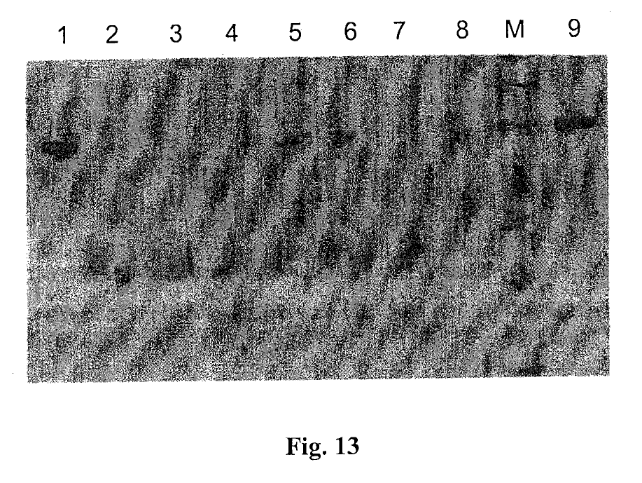

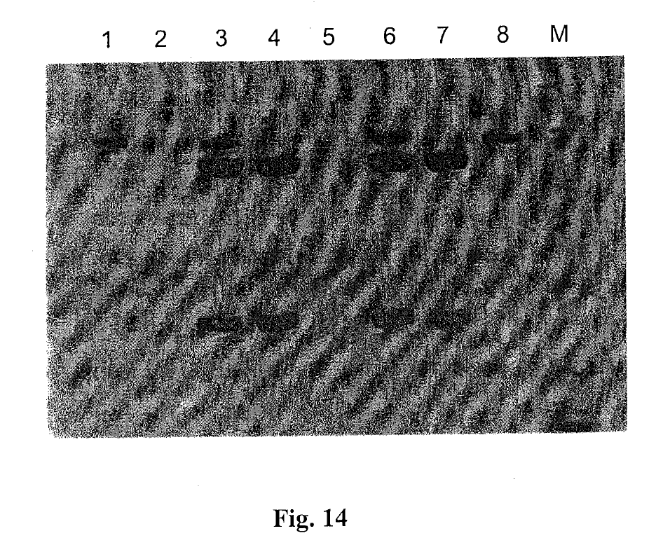

Cleavage of the TGP1 protein by means of MMP9 (Calbiochem) exposure was demonstrated by the appearance of cleavage products of the expected mass in the presence of increasing amounts of recombinant MMP9 both, at room temperature and at 37° C. (FIG. 13). Cleavage of TGP1 also occurred in the presence of Cetuximab suggesting that the presence of antibody did not interfere with accessibility of the protease target site on TGP1 (FIG. 14).

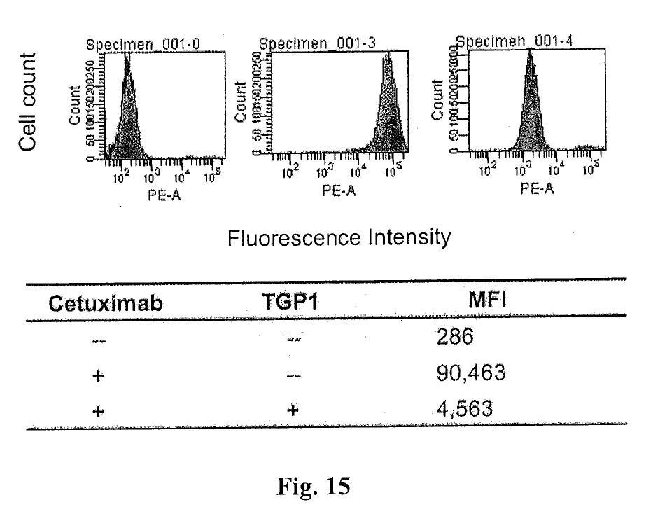

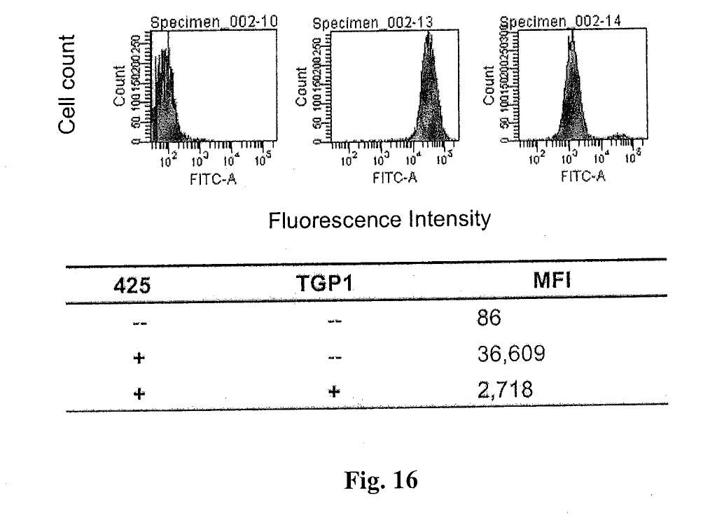

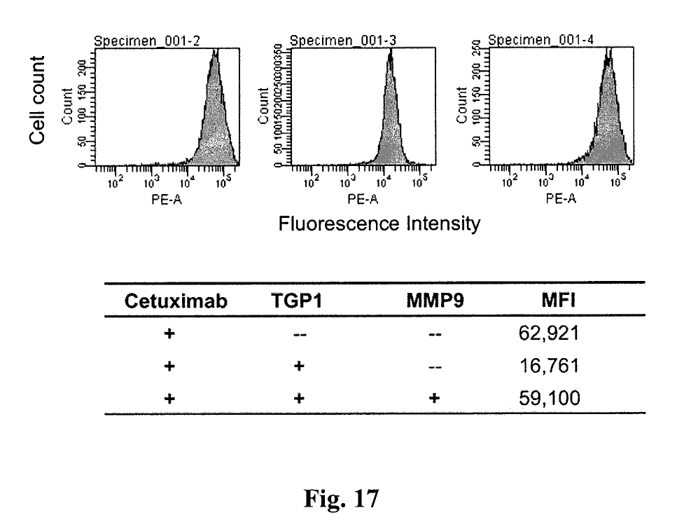

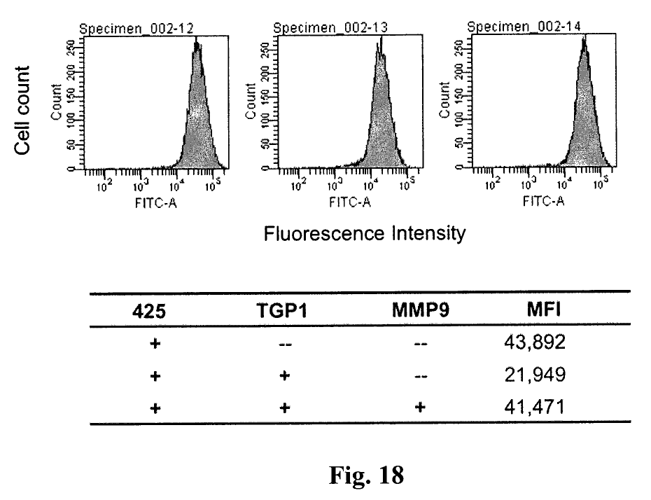

Upon mixing of TGP1 with Cetuximab binding of Cetuximab to breast cancer cells MDA-MB-468 which strongly express the target antigen EGFR was reduced by over 90% (FIG. 15) as determined by FACS analysis and consistent with efficient masking of the Cetuximab CDRs. A similar result was obtained when TGP1 was premixed with the 425 antibody (FIG. 16) although binding of 425 as measured by mean fluorescence intensity was weaker than that of Cetuximab consistent with previously observed overall weaker affinity of 425 for the EGFR (Donaldson et al., Cancer Biol and Ther. 8:2135-40). These experiments were performed at 4° C. and masked and unmasked antibodies incubated with target cells for 30 min. Conversely, addition of MMP9 restored binding of C225 and of 425 to MDA-MB-468 target cells (FIGS. 17 and 18) demonstrating reversible binding inhibition contingent on the presence of proteolytic enzymes. The latter experiments were performed by incubating masked and unmasked antibodies for 2 h at 15° C. with target cells. Collectively, these results demonstrate effective masking and unmasking by MMP9 cleavage of the TGP1 mask.

Collectively, it has been demonstrated that non-covalently bound, multivalent antigenic epitopes (masks) can be used to reversibly occlude antigen binding sites on monoclonal antibodies with therapeutic or diagnostic utility. It has been further shown that reduction to monovalent interaction by cleavage of the multivalent compounds by disease-associated enzymes weakens interaction with the cognate antibodies by reduction to monovalency, leads to disassembly of the complex and, encourages presumably bivalent antigen engagement on target, e.g., tumor cells. These experiments highlight the feasibility of this concept.

The work already accomplished revealed that the presumably bivalent interaction complex between Cetuximab and TGP1 may spontaneously dissociate to some degree. If this should present a problem in vivo, it can be avoided, in part, by constructing very stable tretravalent complexes consisting of “heteromasks,” for example, as outlined in FIGS. 4B-D. In some aspects, point mutations can be engineered into the masks which will favor interaction with two distinct antibodies, for example Cetuximab and Matuzumab, rather than one antibody only. The bivalent nature of either antibody will favor a molecular arrangement in which the second Fab will serve as docking station for a second heteromask thus giving rise to a tetravalent complex in “head-to-head” orientation. The affinity of tetravalent interaction is considerably higher than that of bivalent interaction and will reduce spontaneous disassembly of the complex by decreasing K_{off}. However, protease cleavage of either bivalent or tetravalent complexes will invariably result in reduction to monovalent interaction of a given mask fragment with an individual Fab. In this configuration, bivalent interaction of the antibody molecule with membrane-bound, cell-associated antigen is favored. Similar tetravalent complexes can be constructed using entirely different masks corresponding to different therapeutic antibodies which may be used in combination with therapeutic intent.

A further modification of the mask itself to address problems arising from too low affinity of the mask-antibody complex can be the use of a “generic” non-covalent ligand which recognizes framework residues on the Ig molecule. This can be added to the mask by a peptide linker which may also contain a protease sensitive sequence. In this aspect, the affinity of the uncleaved individual masks is increased by bivalent interaction. However, proteases prevalent at disease sites would lead to not only disruption of the tandem mask complex but also cleave the extension of each individual mask by added framework interacting moieties thus reducing mask affinity to that equivalent to simple monovalent interaction.

These considerations underline the capacity to introduce modifications into the basic design of non-covalent masks to arrive at flexible designs with clinical utility.

### Example 2

### Preparation of a Mask to Conceal the Antigen Binding Site of a mAb and is Chemical Connection of the Mask to a Linker to Form a Masking Ligand

This is a prophetic example. Arecombinant mask can be produced and purified using the recombinant protein expression technologies outlined above and then connected by chemical modification to a second mask via a linker. A reactive group can be added to the N-terminus of the first mask protein via N-terminal transamination, for example as described by Scheck and Francis, ACS Chemical Biology 2: 247-251, 2007; Scheck, et al., J. Am. Chem. Soc. 130: 11762-11770, 2008. The reactive group, a ketone or aldehyde, can then be used to chemically attach the linker protein to the mask proteins.

A linking peptide having the sequence of SEQ ID NO:5, wherein m=1 and n=1, can be chemically synthesized according to standard manufacturing protocols. The linking peptide can contain an alkoxyamine at one end and a reactive sulphur group at the other end. The linker can be coupled to the reactive group on the first mask and the non-reactive linker can be removed by ultrafiltration. The second mask can be engineered to have an N- or C-terminal cysteine. The reactive sulfhydryl group on the linker can be coupled through a bifunctional maleimide reaction to this cysteine moiety.

Polyethylene glycol can be substituted as a linker between the masks and the protease sites. Ethylene glycol units (1 to 10) can be chemically tethered to the N and C-termini of the MMP-9 cleavage peptide, VPLSLYS, to yield NH2-(EG)ₙ-VPLSLYS-(EG)ₘ-NH2, wherein EG is ethylene glycol, and the values of n and m range from 1-10, The amine groups extending from the ethylene glycol can then be converted to oximes through standard chemical procedures. The pryridoxal-5′-phosphate reaction, (described in Scheck and Francis, ACS Chem. Biol. 2: 247-251, 2007), can be applied to create a dialdehyde group on each antibody. The modified masks can then be mixed with the oxime-(EG)ₙ-VPLSLYS-(EG)ₘ-oxime to produce the complete masking ligand (mask-(EG)ₙ-VPLSLYS-(EG)ₘ-mask), as shown in FIG. 5.

The complete multivalent masking ligand can be purified by any appropriate method, such as size exclusion chromatography, ion exchange chromatography, or preparative native gel electrophoresis. The resultant multivalent masking ligand for Cetuximab would have an approximate molecular weight of 72-74 kD.

### Example 3

### Binding the Masking Ligand to the mAb or mAbs

This is a prophetic example. The multivalent masking ligand of Example 2 can be mixed at near stoichiometric amounts with a mAb for Cetuximab in a standard buffer, e.g., PBS, TBS, and allowed to bind to the antibody. The resulting stoichiometry of the masked antibody complex will be 1:1 or 2:2 masking ligand:antibody. Size exclusion chromatography can be used to purify the desired product. The molecular mass of the 1:1 admixture will be approximately 225 kDaltons (antibody is ˜150 kD and masking ligand is ˜75 kD). The molecular mass of the 2:2 admixture will be approximately 450 kD. In this example, the masks of the multivalent masking ligand are identical.

To produce a hetero-masking (non-identical) ligand, distinct antibodies, e.g., anti-Her2 and anti-IGFR, can be mixed with the hetero-masking ligand at a 1:1:2 ratio and allowed to bind with the antibodies. Size exclusion chromatography can be used to purify the masked antibody complex.

Masked antibody complexes can be concentrated to about 2 mg/mL, exchanged into saline buffer, and sterile-filtered for storage at 4° C. Stoichiometry of the complexes can be verified by analytical ultracentrifugation using either sedimentation velocity or sedimentation equilibrium protocols (Kamat, et al. 2008). Masking efficiency can be tested through surface plasmon resonance (SPR) and FACS analysis. Using SPR, the native receptor can be physically coupled to a surface substrate, such as a chip, and calibrated with native antibodies. The isolated and purified masked antibody complex can be passed over the sensor to detect the level of binding. To verify cleavage, the masked antibody complex can be cleaved with the appropriate protease (e.g., MMP-9, which is commercially available). Upon cleavage, the masking ligand becomes monovalent and the therapeutic antibody will partition to the antigen on the chip. This procedure can also be used as a diagnostic for the clinical preparation of masked antibodies. SPR and FACS methodology is known in the art.

Complexes of two or more antibodies can be created by cross-linking the masking ligands of masked antibodies, as shown in FIG. 7. In this way, multiple antibodies can be delivered simultaneously to a target site, increasing efficacy.

### Example 4

### Preparation of Masks with Weaker Affinity for the mAb

This is a prophetic example. Masks with weakened affinity for the antibody can also be prepared to enhance dissociation and binding of the unmasked antibody to antigen in the target tissue. Point mutations can be generated in the mask peptide at the antibody-antigen interface. Most therapeutic antibodies have been isolated from animal sources and then humanized. In general, the host organism (mouse, rat, rabbit, goat, etc.) also encodes a homologous protein. Comparing the sequence of the antigen and the homologous protein from the host provides a limited selection of potential epitopes. However, the selection can be enhanced by comparing sequence differences among organisms on the solvent exposed surface of the antigen. Reversion of the exposed sequence differences to the host sequence helps ensure the structural integrity. Reversion mutants that reduce the binding affinity by 10- to 1000-fold can be used to weaken the affinity of the concealing agent to the therapeutic antibody, as described in Kamat, et al., 2008.

### Example 5

### Masking Antibodies with Generic Masking Ligands

This is a prophetic example. A “generic” masking ligand may be used to mask antigen binding sites irrespective of antigen specificity, as shown in FIG. 8A. A generic epitope (e.g., FLAG® (Sigma-Aldrich) or His tag) followed by a tissue specific protease cleavage sequence (e.g., MMP9) can be genetically added to the N-termini of a therapeutic antibody. The tags bind to a diabody, thereby masking the antigen binding sites of the antibody as shown in FIG. 8A. The mask can be removed by proteolytic cleavage of the proteolytic sites. These generic masking ligands can also be used to connect the framework regions of two antibody molecules, thereby masking their antigen binding sites and forming a tetramer structure as shown in FIG. 8B.

### Example 6

### Delivery of Masked Antibodies to a Target Tissue in a Subject

This is a prophetic example. The masked antibody complexes can be administered intravenously in saline buffer to a mammalian subject in need thereof. Dosage and frequency of administration are determined by the type of antibody and physiology of the subject. It is predicted that the dosage will be approximately 50-500 mg for a 70-80 kg subject.

Upon administration, the antibody complexes are disseminated by the circulatory system and are taken up by tissues. In normal tissue, the antibody complexes remain intact and are not retained. In diseased or tumor tissue, the linker between the masks is cleaved by endogenous proteolytic enzymes, the masks is dissociate from the antibodies, and the antibodies bind to surface antigens on tumor cells. Unbound, masked antibodies are eventually degraded and excreted.

The present invention is not limited to the embodiments described and exemplified above, but is capable of variation and modification within the scope of the appended claims.

1. A masking ligand for reversibly concealing the antigen-binding site of an antibody, comprising two copies of the epitope of the antigen to which the antibody specifically binds and a cleavable polypeptide linker joined to each copy of the epitope.

2. The masking ligand of claim 1, wherein the polypeptide linker comprises at least one proteolytic enzyme recognition site.

3. The masking ligand of claim 2, wherein the proteolytic enzyme is a matrix metalloprotease.

4. The masking ligand of claim 3, wherein the matrix metalloprotease is matrix metalloprotease 9.

5. The masking ligand of claim 1, wherein the polypeptide linker comprises an amino acid sequence selected from the group consisting of SEQ ID NO:5, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.

6. The masking ligand of claim 1, wherein the epitope has at least one amino acid mutation that decreases the affinity of the antibody for the epitope.

7. The masking ligand of claim 1, wherein the antibody specifically binds to epidermal growth factor.

8. The masking ligand of claim 7, wherein the epitope comprises th-e an amino acid sequence selected from the group consisting of SEQ ID NO:7, SEQ ID NO:8, SEQ ID NO:9, or and SEQ ID NO:10.

9. A complex comprising the masking ligand of claim 1 noncovalently bound to the antigen binding site of the antibody.

10. The complex of claim 9, wherein the antibody comprises a detectable label.

11. The complex of claim 9, wherein the antibody comprises at least one post-translational modification.

12. A composition comprising the complex of claim 9 and a pharmaceutically acceptable carrier.

13. A method for preparing a multivalent masking ligand, comprising joining at least two copies of the epitope of an antibody to a cleavable polypeptide linker comprising at least one proteolytic enzyme recognition site.

14. The method of claim 13, wherein each copy of the epitope is chemically modified to include a moiety that can bind to the polypeptide cleavable linker.

15. The method of claim 13, wherein the cleavable polypeptide linker has an amino acid sequence selected from the group consisting of SEQ ID NO:5, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.

16. A method for treating a subject in need thereof, comprising administering to a target tissue in the subject an effective amount of the composition of claim 12, wherein the target tissue expresses a proteolytic enzyme capable of cleaving the cleavable polypeptide linker, and wherein the masking ligand dissociates from the antigen binding site of the antibody at the target tissue after the proteolytic enzyme cleaves the cleavable polypeptide linker.

17. The method of claim 16, wherein the target tissue is a tumor, ulcer, wound, or is inflamed.

18. A masking ligand for reversibly concealing each antigen-binding site of an antibody having more than one antigen binding site with each antigen binding site having a different specificity relative to the other antigen binding sites, comprising a copy of the epitope for each antigen binding site of the antibody and a cleavable polypeptide linker joined to each copy of the epitope.

19. The masking ligand of claim 18, wherein the polypeptide linker comprises at least one proteolytic enzyme recognition site.

20. The masking ligand of claim 18, wherein the polypeptide linker comprises an amino acid sequence selected from the group consisting of SEQ ID NO:5, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.


SEQUENCE LISTING (19 sequences)

SEQ ID NO: 1 - protein, 455 aa, Homo sapiens
SCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQGASSGGGGSGVPLSLYSGGSGGGSVPLSLYSGSTSGSGVPLSLYSKSSEGSGSGAQGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQ

SEQ ID NO: 2 - protein, 455 aa, Homo sapiens
SCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTPGQKTKIISNRGENSCKATGQGASSGGGGSGVPLSLYSGGSGGGSVPLSLYSGSTSGSGVPLSLYSKSSEGSGSGAQGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTPGQKTKIISNRGENSCKATGQ

SEQ ID NO: 3 - protein, 455 aa, Homo sapiens
SCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTSGQKTKIISNRGENSCKATGQGASSGGGGSGVPLSLYSGGSGGGSVPLSLYSGSTSGSGVPLSLYSKSSEGSGSGAQGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTSGQKTKIISNRGENSCKATGQ

SEQ ID NO: 4 - protein, 422 aa, Homo sapiens
SCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTSGQKTKIISNRGENSCKATGQGASSGGGGSGVPLSLYSGGSGGGSSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTPGQKTKIISNRGENSCKATGQ

SEQ ID NO: 5 - protein, 15 aa, Artificial
  Completely Synthesized
SSGSVPLSLYSSSGS

SEQ ID NO: 6 - protein, 51 aa, Artificial
  Completely Synthesized
DYKDDDDKGGGSGGSGSGGGSGGGSVPLSLYSGSTSGSGKSSEGSGSGAQG

SEQ ID NO: 7 - protein, 470 aa, Homo sapiens
MRPSGTAGAALLALLAALCPASRARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECGGGSGGGSGGGSVPLSLYSGSTSGSGKSSEGSGSGAQGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECGGSG

SEQ ID NO: 8 - protein, 470 aa, Homo sapiens
MRPSGTAGAALLALLAALCPASRARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTPGQKTKIISNRGENSCKATGQVCHALCSPEGCWGPEPRDCVSCRNVSRGRECGGGSGGGSGGGSVPLSLYSGSTSGSGKSSEGSGSGAQGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTPGQKTKIISNRGENSCKATGQVCHALCSPEGCWGPEPRDCVSCRNVSRGRECGGSG

SEQ ID NO: 9 - protein, 470 aa, Homo sapiens
MRPSGTAGAALLALLAALCPASRARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECGGGSGGGSGGGSVPLSLYSGSTSGSGKSSEGSGSGAQGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECGGSG

SEQ ID NO: 10 - protein, 470 aa, Homo sapiens
MRPSGTAGAALLALLAALCPASRARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTPGQKTKIISNRGENSCKATGQVCHALCSPEGCWGPEPRDCVSCRNVSRGRECGGGSGGGSGGGSVPLSLYSGSTSGSGKSSEGSGSGAQGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQEGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECGGSG

SEQ ID NO: 11 - protein, 38 aa, Artificial
  Completely Synthesized
GGGSGGGSGGGSVPLSLYSGSTSGSGKSSEGSGSGAQG

SEQ ID NO: 12 - protein, 19 aa, Artificial
  Completely Synthesized
SSGSSSGSVPLSLYSSSGS

SEQ ID NO: 13 - protein, 23 aa, Artificial
  Completely Synthesized
SSGSSSGSSSGSVPLSLYSSSGS

SEQ ID NO: 14 - protein, 19 aa, Artificial
  Completely Synthesized
SSGSVPLSLYSSSGSSSGS

SEQ ID NO: 15 - protein, 23 aa, Artificial
  Completely Synthesized
SSGSSSGSVPLSLYSSSGSSSGS

SEQ ID NO: 16 - protein, 27 aa, Artificial
  Completely Synthesized
SSGSSSGSSSGSVPLSLYSSSGSSSGS

SEQ ID NO: 17 - protein, 23 aa, Artificial
  Completely Synthesized
SSGSVPLSLYSSSGSSSGSSSGS

SEQ ID NO: 18 - protein, 27 aa, Artificial
  Completely Synthesized
SSGSSSGSVPLSLYSSSGSSSGSSSGS

SEQ ID NO: 19 - protein, 31 aa, Artificial
  Completely Synthesized
SSGSSSGSSSGSVPLSLYSSSGSSSGSSSGS